(19) United States
(12) Patent Application Publication
(10) Pub. No.: US 2015/0044710 A1
(43) Pub. Date: Feb. 12, 2015

(54) ENZYMATIC SENSORS AND METHODS FOR THEIR PREPARATION AND USE

(71) Applicant: University of Calcutta, Kolkata (IN)

(72) Inventors: Anjan Kr. DASGUPTA, Kolkata (IN); Sanhita RAY, Kolkata (IN); Arumoy CHATTERJEE, Chinsurah (IN); Tamoghna BHATTACHARYYA, Dist-Birbhum (IN); Satarupa BHADURI, Kolkata (IN); Anirban BOSE, Parganas (IN)

(21) Appl. No.: 14/455,387

(22) Filed: Aug. 8, 2014

(30) Foreign Application Priority Data
Aug. 9, 2013 (IN) 942/KOL/2013

(51) Int. Cl. C12Q 1/66; A61B 5/1455

(52) U.S. Cl. CPC C12Q 1/66; A61B 5/14551
USPC 435/8; 435/288.7; 600/364

(57) ABSTRACT

Disclosed herein are methods, compositions and devices for detecting oxygen in various samples such as environmental and biological samples.

### BACKGROUND

In-line measurement for monitoring molecular oxygen is vital in certain applications to optimize product yield and quality and to ensure process safety. Cutting-edge optical technology provides precise measurement down to trace levels. However, many of such technologies require elaborate chemical or electrochemical techniques.

### SUMMARY OF THE INVENTION

In some aspects, the present technology provides a method of measuring molecular oxygen. In some embodiments, the method includes contacting a first sample with at least one bacterial luciferase composition, wherein the bacterial luciferase composition comprises at least one lipid-functionalized graphene or lipid-functionalized fullerene and at least one luciferase, wherein the luciferase is entrapped within the lipid-functionalized graphene or the lipid-functionalized fullerene, wherein the entrapped luciferase is not linked to the lipid-functionalized graphene or the lipid-functionalized fullerene, and detecting luminescence.

In some aspects, the present technology provides a composition having at least one lipid-functionalized graphene or lipid-functionalized fullerene and at least one luciferase, wherein the luciferase is entrapped within the lipid-functionalized graphene or the lipid-functionalized fullerene, wherein the entrapped luciferase is not linked to the lipid-functionalized graphene or the lipid-functionalized fullerene.

In some aspects, the present technology provides a method to entrap at least one luciferase, at least one associated protein, or a combination thereof. In some embodiments, the method includes combining lipid-functionalized carbon nanotubes and at least one luciferase, at least one associated protein, or a combination thereof to form a mixture and sonicating the mixture.

In some aspects, the present technology provides a kit for making at least one luciferase composition. In some embodiments, the kit includes a first container containing at least one lipid-functionalized graphene or lipid-functionalized fullerene and a second container containing at least one luciferase.

In some aspects, the present technology provides a kit having a container containing at least one luciferase composition, wherein the luciferase composition includes at least one lipid-functionalized graphene or lipid-functionalized fullerene; and at least one luciferase, wherein the luciferase is entrapped within the lipid-functionalized graphene or the lipid-functionalized fullerene, wherein the entrapped luciferase is not immobilized on the lipid-functionalized graphene or the lipid-functionalized fullerene.

In some aspects, the present technology provides a biosensor. In some embodiments, the biosensor include a luciferase composition, wherein the luciferase composition includes at least one lipid-functionalized graphene or lipid-functionalized fullerene and at least one luciferase, wherein the luciferase is entrapped within the lipid-functionalized graphene or the lipid-functionalized fullerene, wherein the entrapped luciferase is not immobilized on the lipid-functionalized graphene or the lipid-functionalized fullerene.

In some aspects, the present technology provides for a device for measuring molecular oxygen. In some embodiments, the device includes at least two removable measuring chambers, wherein the measuring chamber contains a mixture of compounds, wherein the mixture comprises at least one luciferase composition of the present technology and at least two removable vessels, wherein the vessels have a removable top and a needle disposed on a bottom, wherein the vessel and the measuring chamber can be connected by inserting the needle into a top of the measuring chamber.

In some embodiments, the device includes a catheter, an optic fiber, wherein a first end of the optic fiber is within the catheter, a semi-permeable membrane chamber, wherein the semi-permeable membrane chamber is attached on first end of the optic fiber, a mixture of compounds, wherein the mixture comprises at least one luciferase composition of the present technology and wherein the mixture is disposed within the semi-permeable membrane chamber, a photon detector, wherein a second end of the optic fiber is connect to the photon detector, and a processing unit, wherein the processing unit is connected to the photon detector.

### BRIEF DESCRIPTION OF THE DRAWINGS

FIG. 1A shows cultures of V. fischeri grown in capillary tubes open at both ends. The capillary tubes containing 0.5 μl of V. fischeri culture were incubated in the presence of air. From left to right, tubes 1-4 and 6-10 are replicates containing 0.5 μl of an overnight culture of V. fischeri in BOSS medium. Tube 5 (middle tube) is the control (milliQ water).

FIG. 1B shows cultures of V. fischeri grown in capillary tubes capillary tubes open at only one end (one end sealed with wax) containing 0.5 μl of V. fischeri culture incubated in the presence of air. From left to right, tubes 2-10 are replicates containing 0.5 μl of an overnight culture of V. fischeri in BOSS medium. Tube 1 (far left) is the control (milliQ water).

FIG. 2A is a microtiter plate that shows V. fischeri biofilms in liquid culture, exposed directly to air. All wells are replicates. Variation in luminescence is due to variable film growth in the different wells or manual error in aspirating liquid.

FIG. 2B is a microtiter plate that shows V. fischeri biofilms that were coated with glycerol, after aspirating the liquid culture, and exposed to air. All wells are replicates. Variation in luminescence is due to variable film growth in the different wells or manual error in aspirating liquid.

FIG. 3A-F are microtiter plates that show V. fischeri biofilms after incubation in presence of air (1st lane), distilled water (2nd lane), and tap water (3rd lane) for (A) 0 min; (B) 5 min; (C) 15 min; (D) 20 min; (E) 25 min; and (F) 30 min. Variability of luminosity among samples in a given lane on the same plate is likely due to variability in manual aspiration.

FIG. 4 is a microtiter plate that show V. fischeri biofilms exposed to different samples. Well A is the biofilm in culture; Well B is the biofilm plus deoxygenated water; Well C is the biofilm plus milliQ water; and Wells D and E are biofilms coated with vegetable oil.

FIG. 5A-D are wells comparing the luminosity of luciferase in bacterial extract (left wells) to SWCNT entrapped luciferase (right wells) with increasing temperature (20° C., 30° C., 40° C., and 50° C., respectively).

FIG. 6 is a graph showing the temperature tolerance curves of luciferase in bacterial extract (open circles) and SWCNT entrapped luciferase (open squares).

FIG. 7A is a graph showing the measurement of oxygen concentration in non-aerated V. fischeri culture using oxygraph and bioluminescence.

FIG. 7B is a graph showing the normalized intensity of light and oxygraph reading of non-aerated V. fischeri culture against time.

FIG. 7C is a graph showing the normalized log (L) vs. log [O₂] (nmoles/ml) obtained from light and oxygen measurements of non-aerated V. fischeri culture.

FIG. 8 is a non-limiting example of an enzyme composition of the present technology.

FIG. 9 is a schematic of an exemplary device that uses the enzyme compositions of the present technology to measure molecular oxygen levels in a sample.

FIG. 10 is a schematic of an exemplary device that uses the enzyme compositions of the present technology to measure molecular oxygen levels in tissue.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here.

Disclosed herein are compositions and methods related to the manufacture and use of thermostabilized luciferase. In some embodiments, the enzyme compositions and methods disclosed herein include (1) at least one luciferase; and (2) a plurality of lipid-functionalized carbon nanotubes, wherein the luciferase is entrapped by the carbon nanotubes but not linked to the carbon nanotubes. In some embodiments, at least one associated protein is entrapped with the luciferase. In some embodiments, the associated protein is not linked to the luciferase or lipid-functionalized carbon nanotubes.

As used in this specification and the appended claims, the singular forms “a”, “an” and “the” include plural referents unless the content clearly dictates otherwise. For example, reference to “a cell” includes a combination of two or more cells, and the like.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein the term “increased enzyme activity” in the context of luciferase refers to an increased amount of product or activity (for example, light, in the case of luciferase) per unit time as compared to a suitable control enzyme, or a more stable output of product or activity under a give condition as compared to a control. In some embodiments, increased activity of an enzyme, for example luciferase, may be exhibited under “optimal” or “standard conditions” for a particular type of enzyme (for example, standard pH, standard temperature, standard substrate, etc.) as compared to a control enzyme under the same standard conditions. Additionally or alternatively, in some embodiments, increased activity may be exhibited under non-standard conditions for a particular type of enzyme (for example, at a higher or lower temperature, higher or lower pH, non-optimal substrate, etc.) as compared to a control enzyme under the same conditions, or as compared to a control enzyme under standard conditions. By way of example, but not by way of limitation, disclosed herein are luciferase enzymes in contact with, but not liked to, carbon nanotubes, wherein the luciferase enzyme has increased activity as compared to a control luciferase enzyme (for example, the same type of enzyme not in contact with carbon nanotubes, wherein the activity of the control enzyme is evaluated under the same conditions of temperature, buffer, pH, substrate, etc. as the luciferase enzyme in contact with the nanotubes). In some embodiments, increased activity refers to the ability to use the same lipid-functionalized carbon nanotube entrapped luciferase in multiple reactions (e.g., use in more reactions serially than a suitable control enzyme).

As used herein “increased thermal stability” in the context of luciferases refers to an enhancement or increase in structural and/or functional integrity, and/or enzyme activity and/or luminescent activity at a temperature or a temperature range outside the “normal” or “standard” temperature or temperature range for a given luciferase, as compared to a suitable control enzyme. By way of example, but not by way of limitation, in some embodiments of the compositions and methods disclosed herein, luciferases entrapped by carbon nanotubes, but in some cases not linked to the carbon nanotubes, exhibit higher stability and/or activity at about 40° C., or at about 42° C., or at about 44° C., or at about 46° C., or at about 48° C., or at about 50° C., about 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C. or higher, or ranges between any two of these values, as compared to a control luciferase enzyme (for example, a luciferase enzyme not entrapped by lipid-functionalized carbon nanotubes). In some embodiments, increased thermal stability describes a more stable output of product or activity under a give condition (e.g., temperature or temperature range) as compared to a control.

As used herein, “control luciferase” or “control” will have a meaning known to those of skill in the art and which will necessarily depend on the aspect of, for example, luciferase activity or conditions to be evaluated. Typically, a control or control luciferase will be compared to a test luciferase (for example, a luciferase that has been modified or treated in some way). The control and the test luciferase will typically be the same type of luciferase and will be derived from the same source. The control luciferase will not undergo the “modification” or “treatment” (for example, will not be entrapped by carbon nanotubes, or will not be in a compositions comprising lipid-functionalized carbon nanotubes), but will be evaluated for luciferase activity or temperature tolerance under the same conditions as the “modified” or “treated” enzyme. Thus, the effects of the “modification” or “treatment” may be determined. In some embodiments, a “modification” or “treatment” includes entrapping a luciferase with lipid-functionalized carbon nanotubes, wherein the luciferase is not linked to the nanotubes. In some embodiments, the luciferase is in contact with, but not linked to, at least one associated protein.

As used herein, the terms “entrapped luciferase” and “luciferase composition” refer to compositions of the present technology including luciferase entrapped by, but not linked to, carbon nanotubes, lipid-functionalized carbon nanotubes, graphenes, lipid-functionalized graphenes, fullerenes and lipid-functionalized fullerenes. Additional, in some embodiments, entrapped luciferase and luciferase composition refer to compositions of the present technology including luciferase in contact with and/or linked to at least one associated protein, wherein both the luciferase and associated protein are entrapped, but not linked to, carbon nanotubes, lipid-functionalized carbon nanotubes, graphenes, lipid-functionalized graphenes, fullerenes and lipid-functionalized fullerenes.

As used herein, the phrase “but not linked to” refers to a lack of intermolecular bonds that would lead to the immobilization of one linking partner with another linking partner, and/or would create a permanent attachment of one linking partner with another linking partner, and/or would bind one linking partner with the other linking partner. By way of example, but not by way of limitation, “a luciferase entrapped by lipid-functionalized carbon nanotubes but not linked to the lipid-functionalized carbon nanotubes” refers to the lack of or absence of intermolecular bonds that would immobilize the luciferase on the lipid-functionalized carbon nanotubes, create a permanent attachment of the luciferase to the lipid-functionalized carbon nanotubes, or binds the luciferase to the lipid-functionalized carbon nanotubes.

As used herein, the term “linked to” refers to intermolecular bonds that could lead to immobilization of one linking partner with the other linking partner, and/or a permanent attachment of one linking partner with the other linking partner, and/or binding one linking partner with the other linking partner. By way of example, but not by way of limitation, with reference to “an associated protein that may be linked to the luciferase,” an associated protein may be immobilized on the luciferase and/or there is a chemical bond between the luciferase and the lipid-functionalized carbon nanotubes.

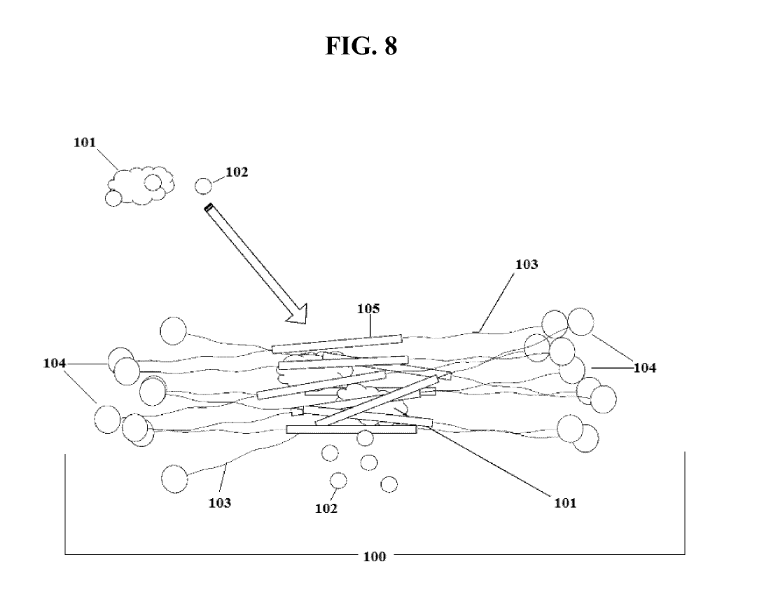

As used herein, “nano-cage” refers to a cage-like structure made of lipid-functionalized carbon nanotubes, graphenes and/or fullerenes. In some embodiments, the nano-cage entraps at least one luciferase. An exemplary, non-limiting, example of a nano-cage is shown in FIG. 8.

As used herein, “associated protein” refers to proteins, polypeptides or enzymes, other than luciferase, that aid in the production and/or amplifyication of luminenscence of the luciferase.

### I. Luciferase Compositions

Disclosed herein are compositions for increasing the enzymatic activity and/or thermostability of luciferase. In some embodiments, at least one luciferase is entrapped in, but not linked to, lipid-functionalized carbon nanotubes, fullerenes and/or graphenes. In some embodiments, the luciferase is in contact with and/or linked to at least one associated protein, wherein both are entrapped in, but not linked to, lipid-functionalized carbon nanotubes, graphenes or fullerenes.

A. Luciferase

Luciferase is a class of oxidative enzymes used in bioluminescence. In some embodiments, luciferases may be isolated from natural sources (for example, from organisms such as bacteria or molds) or may be prepared recombinantly. In some embodiments, the luciferases may be “wild-type” or may be mutant, and may include one or more amino acid substitutions, additions or deletions as compared to the wild-type enzyme. By way of example, but not by way of limitation, in some embodiments, mutations are introduced to produce thermophilic or psychrophilic luciferases. In some embodiments, the luciferase is an isolated bacterial luciferase. Examples of bacterial luciferase include, but are not limited to, Vibrio fischeri, Photobacterium species, Vibrio harveyi, Photobacterium leiognathi, vibrio logei, Photorhabdus sp. and Alteromonas sp.

In some embodiments, the luciferase is a non-bacterial luciferase. Examples of non-bacterial luciferase include, but is not limited to, Renilla luciferase and firefly luciferase.

B. Carbon Nanotubes and Other Carbon Structures

Nanotube based trapping methods of the present technology have a number of advantages over conventional enzyme immobilization methods. By way of example, but not by way of limitation, enzymes of the present technology are suspended in a colloid-like state and the effective surface active area is high. Additionally, nanotube based trapping methods disclosed herein allow for versatile (cross-enzyme) reusability, which conventional immobilization technique do not provide. Easy harvesting of the enzyme after its use makes the methods and compositions disclosed herein economic for the bio-processing activity of choice.

In some embodiments, the luciferase composition includes at least one luciferase is entrapped by, but not linked to, lipid-functionalized carbon nanotubes, graphene and/or fullerenes. As is known in the art, graphene and fullerene are carbon allotropes. Allotropy is the property of some chemical elements to exist in two or more different forms. Graphene, which can be stacked, comprises carbon atoms arranged in a regular hexagonal pattern. Fullerenes are any molecule composed entirely of carbon in the form of a hollow sphere or tube. Example of fullerenes include, but are not limited to, buckyballs (spherical fullerenes) and carbon nanotubes (cylindrical fullerenes).

In some embodiments of the present technology, the luciferase is entrapped by, but not linked to, lipid-functionalized carbon nanotubes. Examples of carbon nanotubes include, but are not limited to, single-wall carbon nanotubes (SWCNT), double-walled carbon nanotubes, or multi-walled carbon nanotubes. In some embodiments, the carbon nanotubes are solid state functionalized with lipids.

Lipids used to functionalize the carbon nanotubes include, but are not limited to, phospholipids, sphingolipids, phosphosphingolipids, and steroids. Typically, there are two fatty acid moieties present on the lipids used in the present technology: a long chain and a short chain.

In some embodiments, the chain length of the short chain lipid is between 2 carbons (“C”) to 10 carbons (“C”) long. In some embodiments the short chain is between 2C and 8C long. In some embodiments, the short chain is 2C, 3C, 4C, 5C, 6C, 7C, 8C, 9C or 10C long. In some embodiments. In some embodiments, the long chain lipid length is 14-22C long. In some embodiments, the long chain is 14C, 15C, 16C, 17C, 18C, 19C, 20C, 21C, or 22C long.

In some embodiments, at least one long chain lipids (for example 22C) will allow the carbon nanotubes to entrap a larger luciferase. For example, at least one longer chain length lipids result in a larger nano-cage. By way of example, but not by way of limitation, FIG. 8 shows an exemplary embodiment of a composition of the present disclosure. The nano-cage of FIG. 8 can entrap larger luciferases but allow smaller complexes/molecules to pass through. By way of example, but not by way of limitation, a luciferase (approximately 80 kDa) and a fatty acid reductase complex (approximately 450 kDa) may be entrapped. Conversely, for example, shorter chain length lipids (for example 14C) result in a tighter nano-cage, which can entrap smaller complexes, but cannot entrap large complexes.

In some embodiments, the ratio of lipid to carbon nanotubes is or about 2:1, about 3:1, or about 4:1, or about 5:1, or about 6:1 by weight.

In some embodiments, the lipid-functionalized carbon nanotube has polar heads on one end of the carbon nanotubes. In some embodiments, the lipid-functionalized carbon nanotube has polar heads on both ends of the carbon nanotubes.

C. Associated Proteins

In some embodiments, the luciferase composition includes one or more associated proteins. In some embodiments, associated proteins aid in the producting and/or amplifying the luminescence of the luciferase. By way of example, but not by way of limitation, in some embodiments, the bioluminescence from the luciferase, due to presence of substrate, excites the associate protein through bioluminescence resonance energy transfer (BRET). In some embodiments, the associated protein is entrapped with the luciferase in the nano-cage, but like the luciferase, the associated protein is not linked to the nano-cage. Additionally, in some embodiments, the associated protein and the luciferase may or may not be linked. Examples of associated proteins include, but are not limited to, NADPH dehydrogenase, lumazine proteins, NADPH-FMN oxidoreductase, yellow fluorescent protein, superoxide dismutase, peroxidases, and fatty acid reductase complex.

### II. Enzyme Activity of Entrapped Enzymes

The entrapment of the luciferase in lipid-functionalized carbon nanotubes results in increased thermostability.

A. Increased Thermostability

In some embodiments, the entrapped luciferase of the present technology has increased thermal stability as compared to a control (not entrapped) enzyme. In some embodiments, increased thermal stability includes a more uniform level of luminescence over a range of temperatures as compared to a control enzyme. By way of example, but not by way of limitation, in some embodiments, the entrapped luciferase maintains luminescent intensity, or a steady level of luminescent intensity at higher temperatures, or over a temperature range, as compared to a control luciferase. At high temperatures, for example, greater than 50° C., the luminescence intensity of free luciferase drastically falls because of significant loss of enzyme activity. Free luciferase will typically lose all enzymatic activity at about 50° C.-60° C. In some embodiments, the present technology increases and/or stabilizes luciferase's enzymatic activity, which results in more luminescence at temperatures at or above about 50° C.-65° C., 50° C.-55° C., 55° C.-60° C., 65° C., 70° C. or higher, as compared to a control (e.g., untrapped) luciferase.

In some embodiments, the increased thermal stability of entrapped luciferase refers to maintaining luciferase activity at a temperature range of about 30° C. to about 70° C., or about 35° C. to about 65° C., or about 55° C. or higher. In some embodiments, the reference temperature in the context of thermal stability of entrapped luciferase is about 30° C., 33° C., 36° C., 39° C., 42° C., 45° C., 48° C., 50° C., 53° C., 55° C., 56° C., 57° C., 58° C., 59° C., 60° C., 65° C., 70° C., 75° C. or higher, or ranges between any two of these values.

### III. Methods for Making Luciferase Composition

In some embodiments, the formation of a luciferase composition of the present technology includes sonicating lipid-functionalized carbon nanotubes, fullerenes and/or graphenes and combining the sonicated mixture with at least one luciferase. The lipid-functionalized carbon nanotubes (or graphenes or fullerenes) will self-assemble into a nano-cage after sonication. The formation of the nano-cage entraps the luciferase. In some embodiments, a liquid culture from luciferase producing bacteria is combined with the sonicated mixture. In some embodiments, the carbon nanotubes are SWCNT. By way of example, but not by way of limitation, entrapped luciferase compositions of the present technology can be formed as follows.

### Exemplary Functionalization Procedure

In some embodiments, SWCNT and lipids are allowed to interact in solid state in a glass close capillary interface. In some embodiments, lipids are allowed to interact with SWCNT both from one end and two ends. A molecular re-structuring occurs at the interface of SWCNT and lipids because of strong hydrophobic interaction between nanotube cores with the long fatty acid tail of the lipid. The solid state chemistry between SWCNT and lipids has been elaborately discussed by Bhattacharyya et al., Nanotechnology (2012) 385304 (8 pp).

### Exemplary Entrapment Procedure

When the SWCNT are subjected to interact with a lipid moiety from one or from the both ends, the linear hydrophobic tail of the lipid moieties enter into the inner hydrophobic core of SWCNT. Accordingly, the complex may be either a SWCNT tail with a polar head group of lipid or SWCNT tail with two polar head groups of lipid. When this complex is subjected to an aqueous environment, the complex becomes water soluble and the hydrophobic portions of the complex assemble with each other to form a cage-like structure. The cage-like structure collapses on mild sonication. In some embodiments, proteins of interest (e.g., luciferase, and in some embodiments, associated proteins) are added to the sonicated mixture. The cages are allowed to re-assemble, thereby entrapping the protein molecules within cage-like structures.

Referring to FIG. 8, in some embodiments, the luciferase composition has carbon nanotubes 105 that are functionalized by lipids 103. In some embodiments, the lipids have polar head groups 104.

In some embodiments, the length of the lipid chain determines the size of the entrapped protein 101 and 102. A longer lipid chain length will entrap a larger protein 101 allowing a smaller protein 102 to “leak out.” In some embodiments, nano-cages are formed with lipid chains of about 14C-22C. In some embodiments, longer lipid chain include a lipid chain length of about 22C.

In some embodiments, shorter lipid chains are used to entrap smaller molecules, such as smaller (for example, lower molecular weight) luciferases. Additionally, nano-cages with shorter lipid chains will prevent larger molecules or substrates from entering the nano-cage. In some embodiments, the smaller nano-cages have a lipid chain length of about 14C.

In some embodiments, the ratio of lipid-functionalized carbon nanotubes to luciferase is about 1:3 to about 1:5 by weight.

In some embodiments, the lipid-functionalized carbon nanotubes are modified to adsorb or scavenge free oxygen radical species (for example, O. or O₂.⁻). Examples of adsorbing agents include, but are not limited to, melanin particles (Cope), semi-conductor particles like SWCNTs themselves, and any particle having charge carrier properties. Examples of scavenging agents include, but are not limited to, anti-oxidants, vitamin C, vitamin E, glutathione, or a combination thereof.

In some embodiments, the adsorbing agent, scavenging agent or a combination thereof is linked to the outer surface of the nan-cage. In some embodiments, the adsorbing agent, scavenging agent or a combination thereof is covalently linked to the outer surface of the nano-cages. In some embodiments, the adsorbing agent, scavenging agent or a combination thereof is entrapped within the nano-cage, wherein the adsorbing agent, scavenging agent, or a combination thereof may or may not be linked to the nano-cage. In some embodiments, the adsorbing agent, scavenging agent or a combination thereof is added to the mixture of sample and luciferase composition.

### IV. Methods of Using Luciferase Compositions

In one embodiment, the present technology is directed at detecting available molecular oxygen (O₂) in a liquid sample, a gel sample, or a gas sample. Luciferases are known to use oxygen as a substrate to generate luminescence. Some luciferases can use molecular oxygen (O₂) and/or oxygen radicals (for example, O. or O₂.⁻) as substrates. Some luciferases, for example, firefly luciferase, will use both molecular oxygen and oxygen radicals as substrates. Bacterial luciferase, e.g., V. fischeri, uses only molecular oxygen as a substrate.

In some embodiments, the entrapped luciferase detects molecular oxygen, dissolved molecular oxygen, or a combination thereof.

In some embodiments, the entrapped luciferase detects molecular oxygen, dissolved molecular oxygen, oxygen radicals, or a combination thereof.

In some embodiments, the detection of dissolved molecular oxygen or molecular oxygen includes contacting a liquid sample, a gel sample, or a gas sample with an entrapped luciferase composition. Alternatively, or additionally, in some embodiments, a mixture of sample and entrapped luciferase is sonicated. The sonication releases the luciferase to generate bioluminescence.

In some embodiments, the contacting is performed at a temperature of about 30° C. to about 70° C., or about 35° C. to about 65° C., or about 55° C. or higher. In some embodiments, the reference temperature in the context of thermal stability of entrapped luciferase is about 30° C., 33° C., 36° C., 39° C., 42° C., 45° C., 48° C., 50° C., 53° C., 55° C., 56° C., 57° C., 58° C., 59° C., 60° C., 65° C., 70° C., 75° C. or higher, or ranges between any two of these values.

In some embodiments, the entrapped luciferase has unattenuated enzyme activity when used in multiple reactions (e.g., multiple serial reactions) as compared to a control luciferase. A non-limiting example of the multiple-reaction use of entrapped luciferase is as follows: 1) at least one entrapped luciferase is contacted with one sample for the duration of a first reaction process; 2) after the completion of the first reaction process, the entrapped luciferase is collected by centrifugation at 5000 g for 10-15 minutes at temperatures in which the enzyme will not be denatured (e.g., 4° C.), entrapped assembly occurs in the pellet; and 3) the collected entrapped luciferase is then re-used (after re-suspension in an appropriate reaction buffer) in a second reaction process. During the second reaction process, the entrapped enzyme maintains enzymatic activity, as measured by intensity of luminescence. In some embodiments, the entrapped luciferase is used in one reaction, or two reactions, or three reactions, or four reactions.

In some embodiments, the entrapped luciferase is collected after a reaction with a substrate by centrifugation at 5000 g for 10-15 minutes at about 4° C.

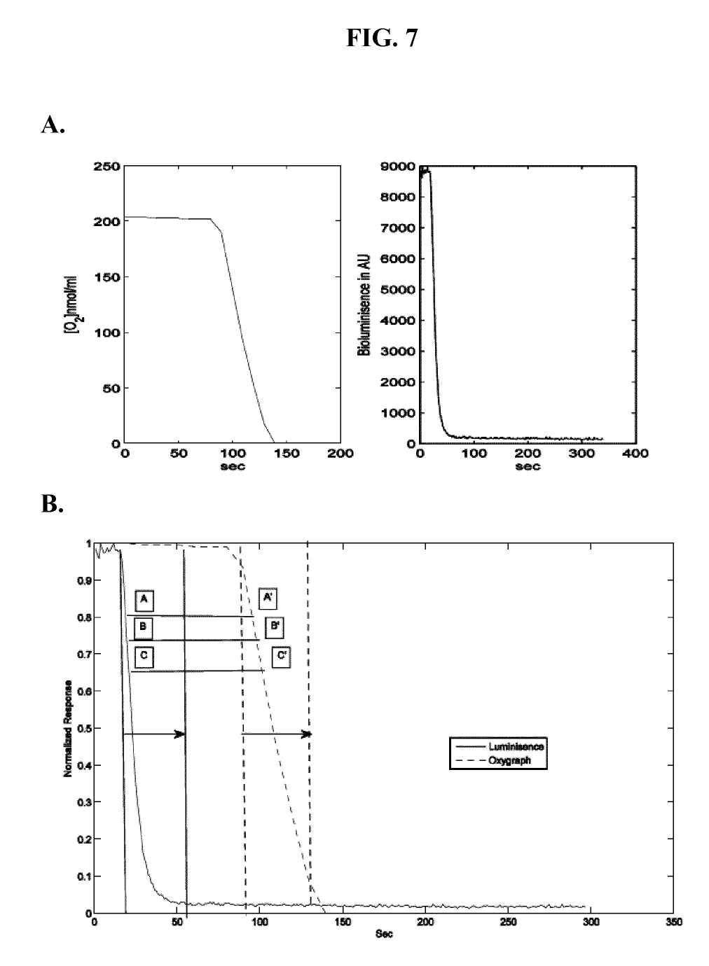

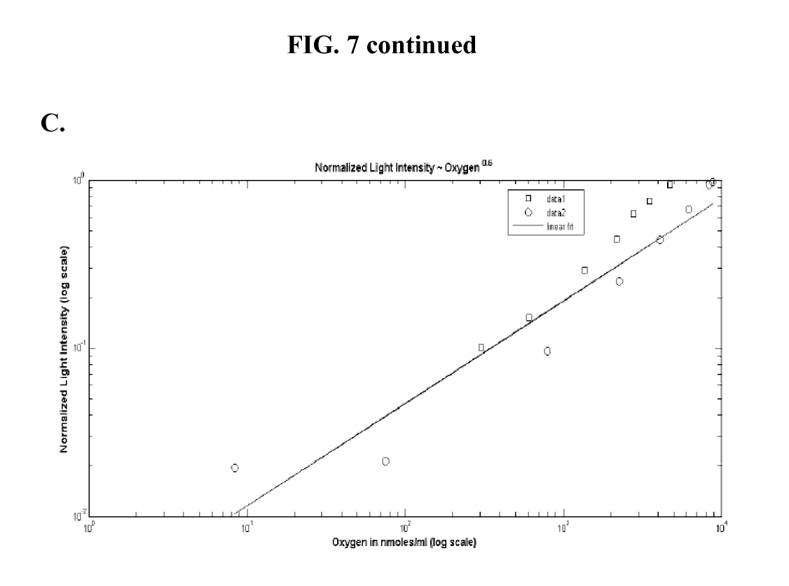

In some embodiments, measuring molecular oxygen comprises calibrating the luminescence against an oxygraph reading (FIGS. 7-8), using electrode based methods (e.g., Clarke electrode), using REDFLASH-dyes, or using optical oxygen sensing based on ruthenium and porphyrin complexes.

### V. Kits

In some embodiments, the luciferase composition is presented in a kit. In some embodiments, the luciferase composition includes at least one luciferase entrapped in lipid-functionalized carbon nanotubes, fullerenes and/or graphenes. In some embodiments, the luciferase is in contact with, but is not linked, the lipid-functionalized carbon nanotubes, fullerenes and/or graphenes. In some embodiments, at least one luciferase in entrapped by lipid-functionalized carbon nanotube, graphene or fullerene, or a combination thereof. In some embodiments, the luciferase is in contact with a lipid-functionalized nanotube, graphene or fullerene, but is not linked to the graphene or fullerene.

In some embodiments, the luciferase composition includes at least one associated protein. In some embodiments, the associated protein is in contact with the luciferase, and may be linked to the luciferase. In some embodiments, the associated protein is in contact with but not linked to the lipid-functionalized nanotube, graphene or fullerene. In some embodiments, the nano-cages of the luciferase composition are modified to scavenge or absorb or adsorb oxygen radicals.

In some embodiments, the kit includes a first container having a plurality of lipid-functionalized carbon nanotubes, graphenese or fullerenes, and a second container having at least one luciferase. In some embodiments, the first container includes a plurality of lipid-functionalized carbon nanotubes, graphenes or fullerenes.

In some embodiments, the kit includes a third container of at least one associated protein. In some embodiments, the kit includes a fourth container having an absorbing agent, an adsorbing agent, a scavenging agent or a combination thereof. In some embodiments, the kit also includes instructions to create a luciferase composition.

In some embodiments, the luciferase is a bacterial luciferase. In some embodiments, the bacterial luciferase includes, but is not limited to one or more of Vibrio fischeri, Photobacterium species, Vibrio harveyi, Photobacterium leiognathi, Vibrio logei, Photorhabdus sp. and Alteromonas sp.

In some embodiments, the luciferase is a non-bacterial luciferase. In some embodiments, the non-bacterial luciferase includes, but is not limited to one or more of Renilla luciferase and firefly luciferase.

In some embodiments, the luciferase is a recombinant luciferase.

In some embodiments, the carbon nanotubes include, but are not limited to one or more of single-wall carbon nanotubes (SWCNT), double-walled carbon nanotubes, or multi-walled carbon nanotubes. In some embodiments, the carbon nanotubes are solid state functionalized with lipids.

In some embodiments, the lipids used to functionalize the carbon nanotubes include, but are not limited to one or more of phospholipids, sphingolipids, phosphosphingolipids, and steroids.

In some embodiments, the chain length of one or more lipid chains is between 2C to 22C long, or between 4C to 20C long, or between 6C to 18C long, or between 8C to 16C long, or between 10C to 14C long. In some embodiments, short chain lipid lengths are 2C, 3C, 4C, 5C, or 6C. In some embodiments, long chain lipid lengths are 14C, 15C, 16C, 17C, 18C, 19C, 20C, 21C, or 22C long.

In some embodiments, the associated proteins include, but are not limited to one or more of NADPH dehydrogenase, lumazine proteins, NADPH-FMN oxidoreductase, yellow fluorescent protein, superoxide dismutase, peroxidases, and fatty acid reductase complex.

In some embodiments, the absorbing agents include, but are not limited to one or more of melanin particles (Cope), semi-conductor particles like SWCNTs themselves, and any particle having charge carrier properties. Examples of scavenging agents include, but are not limited to one or more of anti-oxidants, vitamin C, vitamin E, glutathione, or a combination thereof.

### VI. Illustrative Uses of the Luciferase Compositions Disclosed Herein

A. Biosensors

The composition of the present technology can be used for a variety of purposes. In some embodiments, the present technology is used in the context of a biosensor. In some embodiments, the biosensor is used to detect molecular oxygen in clinical and/or environmental samples and further monitor conditions such as disease state, and/or environmental quality and safety.

In some embodiments, the biosensor is an optode. An optode is an optical sensor device that optically measures a specific substance usually with the aid of a chemical transducer.

B. Devices Using Luciferase Compositions (e.g., Optodes)

In some embodiments, the luciferase compositions of the present technology are used in devices that monitor or determine oxygen levels in a sample or in a tissue. In some embodiments, the device comprises a hand-held device.

In some embodiments, the device comprises a micro-fluidic device. In some embodiments, the luciferase composition is provided within channels of a micro-fluidic device. The size constraints designed into such devices can be used to trap the luciferase composition within the microfluidic channel. Such micro-fluidic devices may be used for multiplexed, real-time measurement of dissolved oxygen from multiple sources.

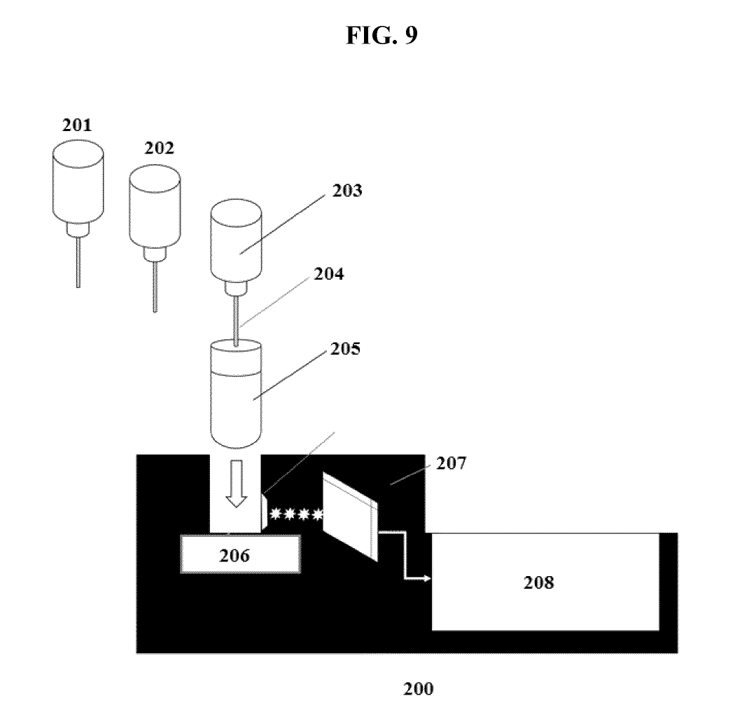

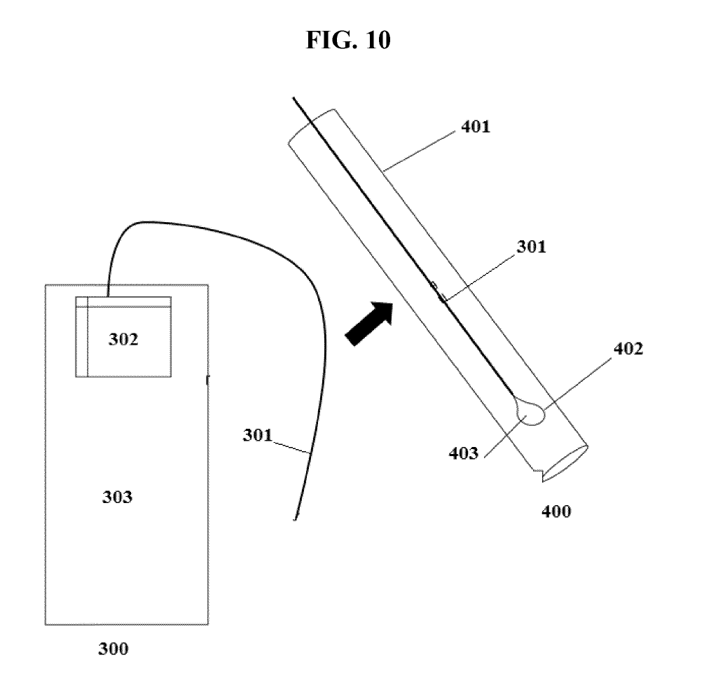

By way of example, but not by limitation, FIGS. 9 and 10 are exemplary embodiments of devices for measuring molecular oxygen using the luciferase composition.

Referring to FIG. 9, in some embodiments, an oxygen measuring device 200, includes at least one air-tight vessels 201-203, and a measuring chamber 205, wherein the measuring device includes a sample holder 206, a photon detector 207, and a display/signal processor unit 208. In some embodiments, the sample holder includes a transparent lens. In some embodiments, the oxygen measuring device 200 includes a housing that holds the sample holder 206, photon detector 207, a display/signal processor unit 208, and the measuring chamber. In some embodiments, the measuring chamber is removable.

Referring to FIG. 9, in some embodiments, the air-tight vessels may include at least one air-tight vessel containing decanal (a luciferase substrate—positive control) 201, at least one air-tight vessel containing deoxygenated milliQ water (negative control) 202, and at least one air-tight vessel used for sample collection 203. In some embodiments, each air-tight vessel includes a needle 204, which is used for channeling the contents of the vessels into the measurement chamber 205.

In some embodiments, the measurement chamber 205 contains dry assay components. In some embodiments, the dry assay components include, but are not limited to, at least one luciferase composition (e.g., semi-solid, lipid modified), NADPH, FMN, and buffer, e.g., mono- and bi-phosphate salts. In some embodiments, the dry assay components in the measurement chamber 205 are under vacuum.

The needle 204 of an air-tight vessel 201-203 is inserted into the measurement chamber 205, wherein the sample in the air-tight vessel is transferred into the measurement chamber 205. In some embodiments, a different measuring chamber 205 is used for each air-tight vessel. In some embodiments, the samples in the air-tight vessels are liquid. The sample and dry assay components are mixed and the measurement chamber 205 is placed in the sample holder 206. In some embodiments, the sample holder 206 includes a transparent lens for focusing light formed in the measurement chamber 205. In some embodiments, the measuring chamber is completely made of a clear material, e.g., glass or clear plastic. In another embodiment, the measuring chamber is made with an opaque material with a transparent area near the transparent lens. In some embodiments, the light is focused onto a photon detector 207. In some embodiments, the photon detector is a CCD array. In some embodiments a display/signal processor unit 208 are connected to the photon detector 207. In some embodiments, the display/signal processor unit 208 measures the light intensity to determine the amount of luminescence, and correlates the light intensity or luminescence to the amount of molecular oxygen in the sample. In some embodiments, the light intensity of the sample is measure against controls to determine molecular oxygen concentration.

Referring to FIG. 10, in some embodiments, the oxygen measuring device is a hand-held device. In some embodiments, the hand-held device 300 includes a processing and display unit 303, which contains a photon detector 302, and an optical fiber 301, which is connect on one end to the photon detector 302 and the other end is disposed in a catheter 400. In some embodiments, the catheter 400 includes a protective sheath 401, the other end of the optical fiber 301, wherein a chamber made of a semi-permeable membrane 402 is attached to the end of the optical fiber. In some embodiments, the chamber 402 contains a luciferase mixture 403, which includes, but is not limited to, at least one luciferase composition, buffer constituents, FMN, NADPH, and decanal.

In some embodiments, the catheter 400 is inserted in a sample or tissue. The molecular oxygen in the sample or tissue react with the luciferase mixture 403 in the chamber 402. In some embodiments, the chamber 402 is removable, which allows attachment of a new chamber containing the luciferase mixture. The reaction between the molecular oxygen and luciferase mixture emits a light which is carried by the optical fiber 301 to the photon detector 302. The processing and display unit 303 measures the intensity of the light emitted to determine the amount of molecular oxygen in the sample or tissue. In some embodiments, the light intensity of the sample is measure against controls to determine molecular oxygen concentration.

In some embodiments, the hand-held oxygen measuring device 300 includes a housing that holds the photon detector 302 and processing and display unit 303.

### EXAMPLES

The present invention is further illustrated by the following examples, which should not be construed as limiting in any way.

### Example 1

### Detection of Oxygen from Liquid Culture in Capillaries Using Vibrio fischeri Luciferase

The following experiment demonstrates that V. fischeri bioluminescence depends on the presence of molecular oxygen.

### Method

V. fischeri MJ 11 strain was stored as glycerol stock (−80° C.) and revived for 24 hours in BOSS medium (30 g NaCl, 10 g peptone, 3 g beef extract, 1.6 ml glycerol, 50 ml Tris buffer, pH 7.3, 950 ml distilled water) at 25° C.

Two sets of thin capillaries were prepared. Set 1 included capillaries open at both ends with 0.5 μl of liquid culture from the V. fischeri confined in them. Set 2 included capillaries containing equal volumes and dilutions of liquid culture as Set 1, but one end of the capillaries were sealed with molten wax to cut off aerial oxygen. Control sets contained the same volume of Boss media. Bioluminescence was measured in Bio-Rad ChemiDoc (Chemiblot Mode, Bio-Rad Protean II X1 gel, 100 seconds exposure) and the bioluminescence difference was observed.

### Results

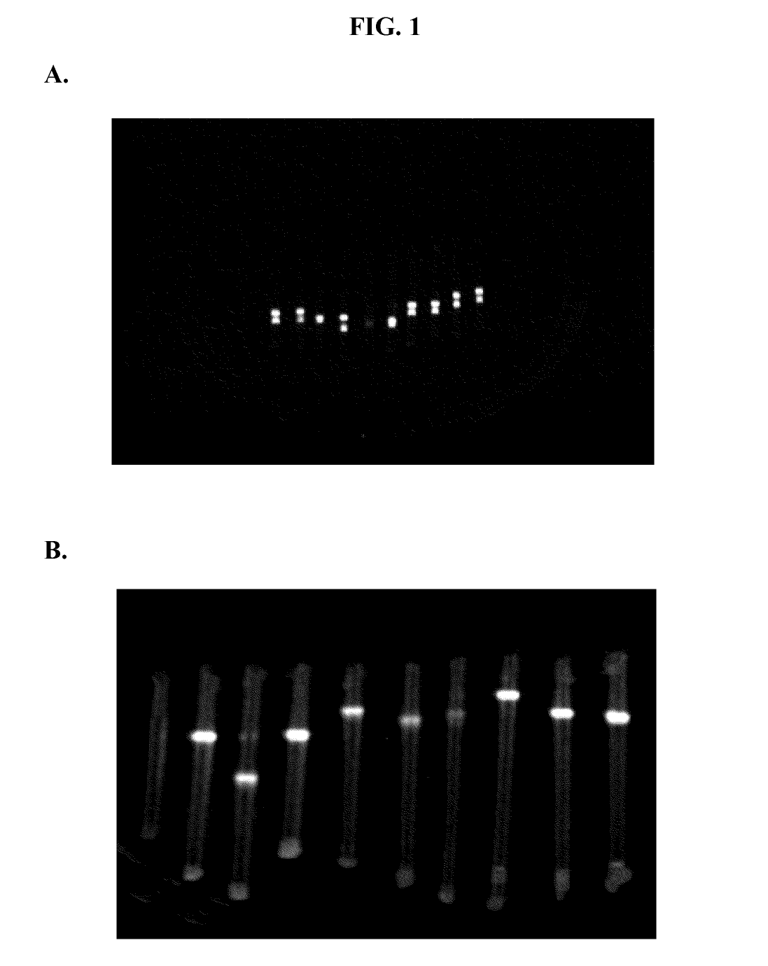

The bioluminescence property of V. fischeri entirely depends on the presence of molecular oxygen. The intensity of the light is dependent on the amount of free molecular oxygen available as the enzyme luciferase uses oxygen as a substrate to produce water. The interior of the culture inside the capillaries did not show luminescence due to absence of oxygen. In contrast, the end positions show greater luminescence as the ends are exposed to aerial oxygen of the atmosphere as shown in FIGS. 1A and 1B respectively.

The results indicate that bacterial luciferases are useful in detecting molecular oxygen. In particular, these results show that the luciferase compositions of the present technology may be useful in detecting molecular oxygen.

### Example 2

### Detection of Oxygen Using Vibrio fischeri Luciferase Bacterial Bio-Films

The following experiment demonstrates the use of V. fischeri bio-films to detect the presence of oxygen.

### Methods

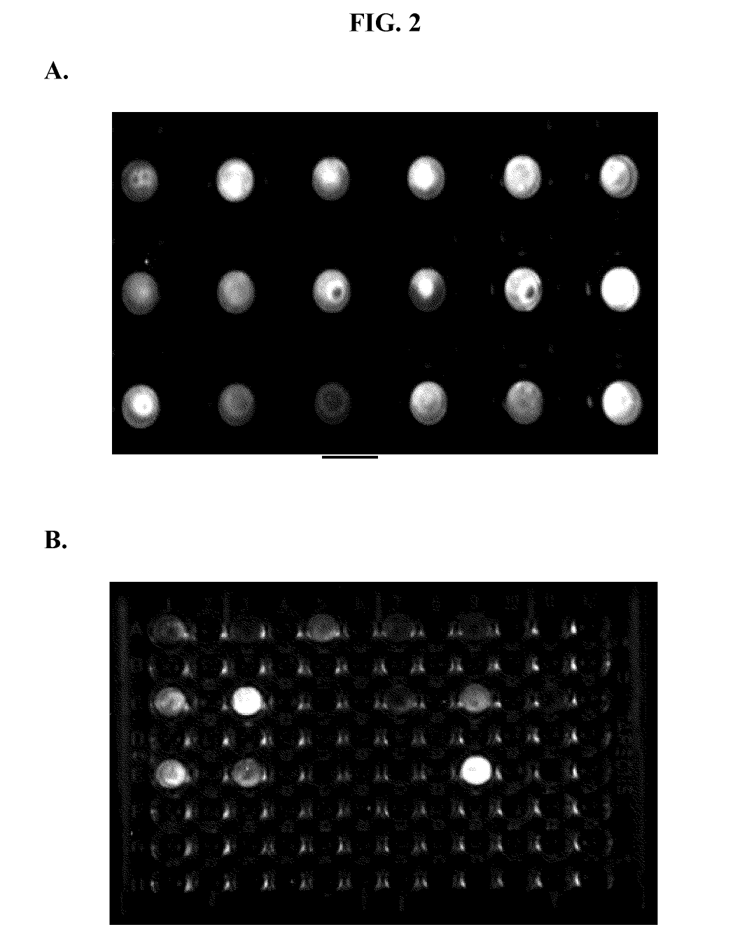

30 μl of liquid culture of V. fischeri was added to the wells of two 96-well-microtiter plate. The plates were incubated for 48 hours at 25° C. to allow biofilm formation. The liquid media from one plate was aspirated from the respective wells and 50 μl of glycerol was added to the respective wells to cover the biofilm surface to block aerial oxygen. Both plates were then exposed to air. The microtiter plates were observed under Bio-Rad ChemiDoc (Chemiblot Mode, Bio-Rad ProteanII X1 gel, 100 seconds exposure). Results are shown in FIGS. 2A and 2B.

In an additional assay, 30 μl of liquid culture of V. fischeri was added to the wells of 96-well-microtiter plate. The plate was incubated for 48 hours at 25° C. to allow biofilm formation. The liquid media from both plates was aspirated from the respective wells and biofilm was retained. Biofilms in lane 1 were exposed to air, biofilms in lane 2 were exposed to distilled water, and biofilms in lane 3 were exposed to tap water. The microtiter plates were observed under Bio-Rad ChemiDoc (Chemiblot Mode, Bio-Rad ProteanII X1 gel, 100 seconds exposure). The change in bioluminescence of the wells was monitored over 30 minutes, with readings taken at 0, 5, 15, 20, 25, and 30 minutes.

### Results

Referring to FIGS. 2A and 2B, when glycerol was added to the biofilm (FIG. 2B), the intensity of luminescence was drastically reduced due to lack of aerial oxygen (compare FIG. 2A with 2B). The thin film of the highly viscous fluid generated on the culture by the glycerol prevented oxygen from contacting the enzyme. The micro-titer plate with biofilm not coated by glycerol had a greater intensity of emitted light because of the exposure to aerial oxygen, FIG. 2A.

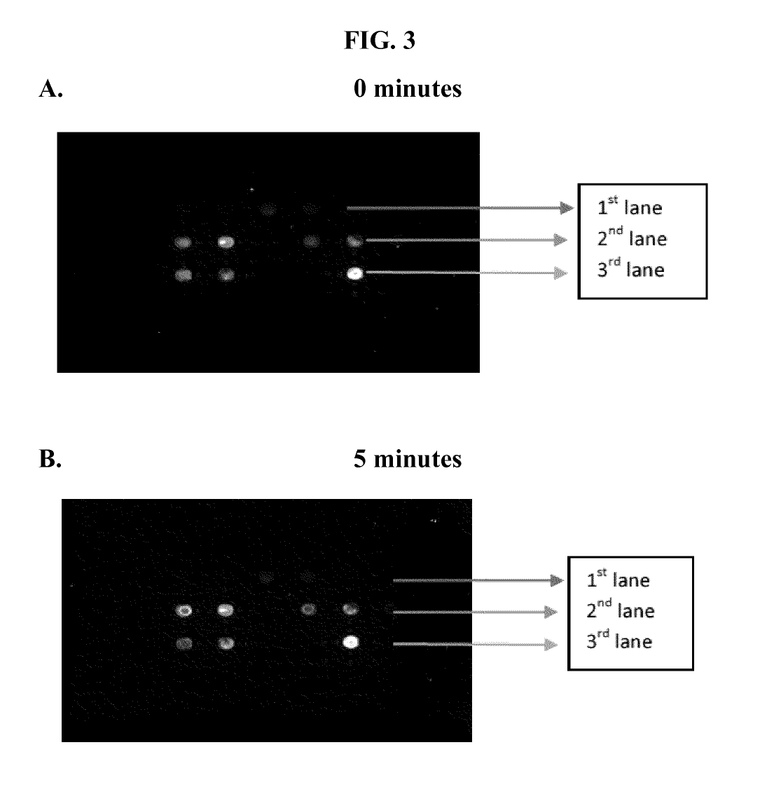

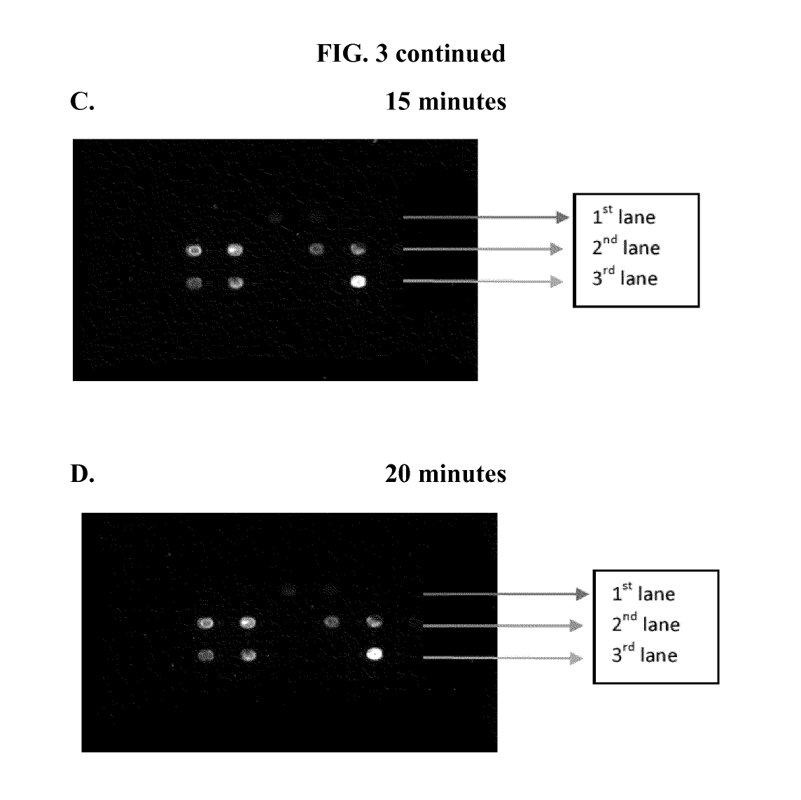

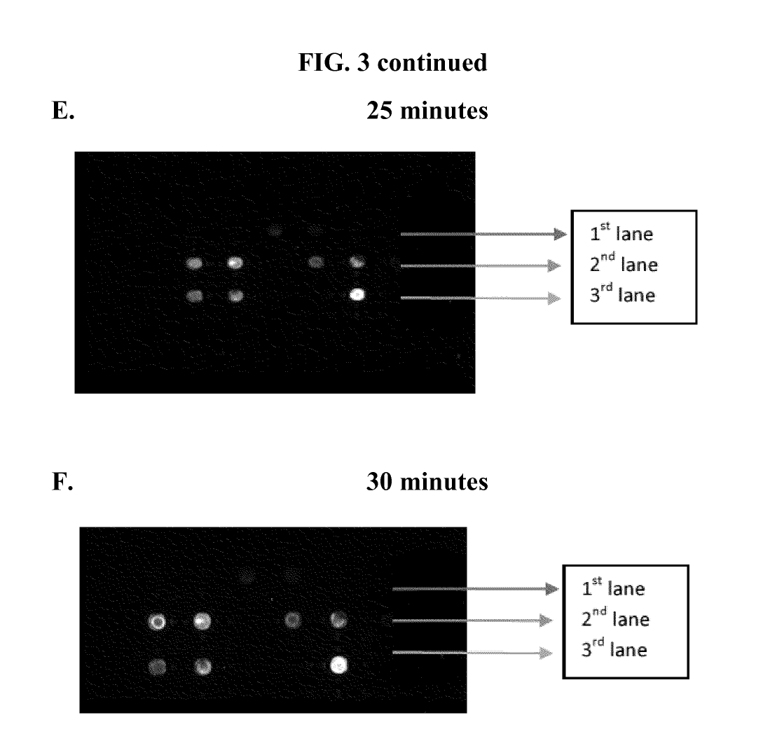

The same light signature was seen after incubation in oxygenated water for 0 minutes, 5 minutes, 15 minutes, 20 minutes, 25 minutes, and 30 minutes, FIG. 3A-3F. Lanes 2 and 3 of the microtiter plate, which contained distilled water and tap water, respectively, displayed intense light (FIG. 3A-3F) because of the dissolution of aerial oxygen in the water sample, while the intensity of light in lane 1 was greatly diminished since the wells were aspirated and had no liquid medium for dissolved aerial oxygen. Well A is a control (biofilm in culture).

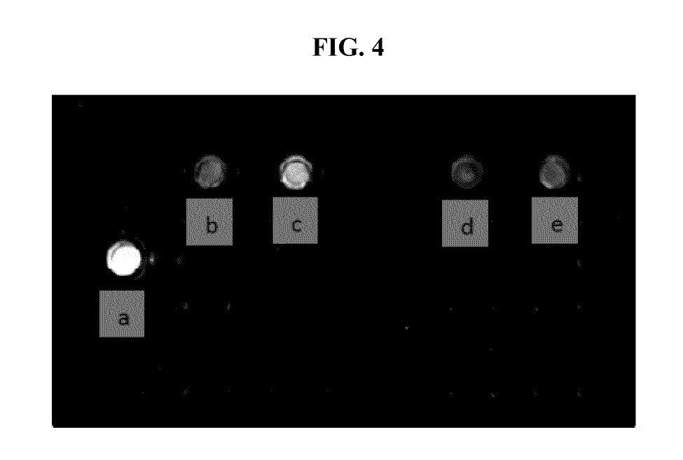

The above experiment was repeated using vegetable oil instead of glycerol with deoxygenated water and miliQ water. FIG. 4 shows that the intensity of luminescence is highest in miliQ water well (Well C) in comparison with the deoxygenated water well (Well B) and microbial culture plus glycerol well (Wells D and E). Again, the loss in light intensity is due to unavailability of dissolved oxygen in water samples.

The results indicate that bacterial luciferases are useful in detecting dissolved molecular oxygen. In particular, these results show that the luciferase compositions of the present technology may be useful in detecting dissolved molecular oxygen.

### Example 3

### Increased Temperature Tolerance of Luciferase Entrapped in Lipid-Functionalized Carbon Nanotubes

Experiments were performed to determine the differences in light production (fixed time assay) between luciferase extract and entrapped luciferase (extract entrapped within lipid-functionalized SWCNT based nano-cage), at temperatures of 20, 30, 40 and 50 degree Celsius, respectively. Light production was measured by ChemiDoc. The assays were performed with cell free extracts prepared from V. fischeri liquid cultures.

Luciferase was entrapped in lipid functionalized nanotube cages as follows. Nanotube functionalization is performed as described by Bhattacharyya et al., Nanotechnology, 23 (2012) 38534 (8 pgs), IPO 529/KOL/2012 and PCT/IB2012/001509. Two nano-surfaces are allowed to interact in a close capillary. Due to solid state interaction, the interface will diffuse only if the reaction occurs at the interface region. A molecular re-orientation is expected at the interface. Such re-orientation is considered as a nano-scale reaction that would be coupled to a diffusive or translocation mechanism, like the solid state chemical reaction.

Lipids are allowed to interact with SWCNT at both ends of the tube. A molecular re-orientation occurs at the interface of SWCNT and lipids. These complexes form a pseudo-micellar structure in water or buffer, e.g., 100 mM phosphate buffer (pH 7.2).

Luciferase is added to the lipid-functionalized SWCNT complex at 40° C. The mixture is sonicated for two minutes at 40° C. After sonication, the lipid-functionalized SWCNT assemble into nano-cages, which entrap the luciferase. In some embodiments, the assembly of nano-cages is performed at 40° C. for 48 hours.

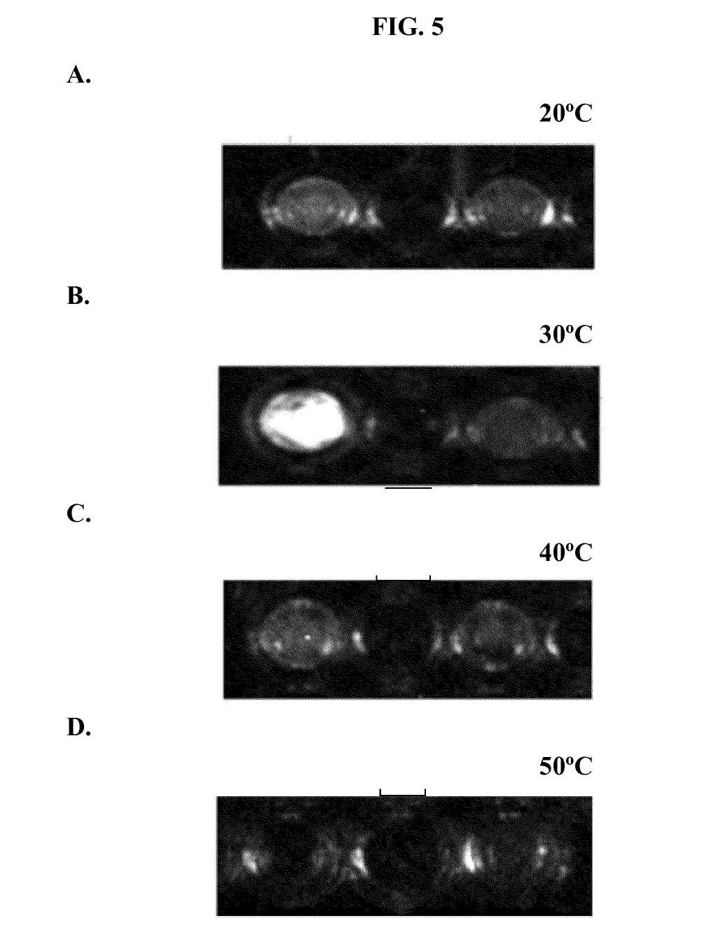

The luminescence intensity of entrapped luciferase enzyme within lipid-functionalized SWCNT was tested at 20° C., 30° C., 40° C. or 50° C. Luminescence intensity is less in comparison with the free luciferase, FIG. 5. As the net amount of entrapped luciferase is much less than the amount of free luciferase, the entrapped luciferase luminescent intensity decreases in comparison with the entire sample of free luciferase luminescence. Additionally, the nano-cage serves as a porous black body, which leads to decreased luminescence due to the surface absorption by the large inner surface area of the nanotube mesh.

When sonicated, the entrapped luciferase is released from nano-cage-entrapment, and the luminence intensity increases, which indirectly confirms that the luciferase enzyme is not linked to the SWCNT. When then nano-cage self-assembles again and entraps the luciferase, the observed luminescence is similar to the original observed luminescence.

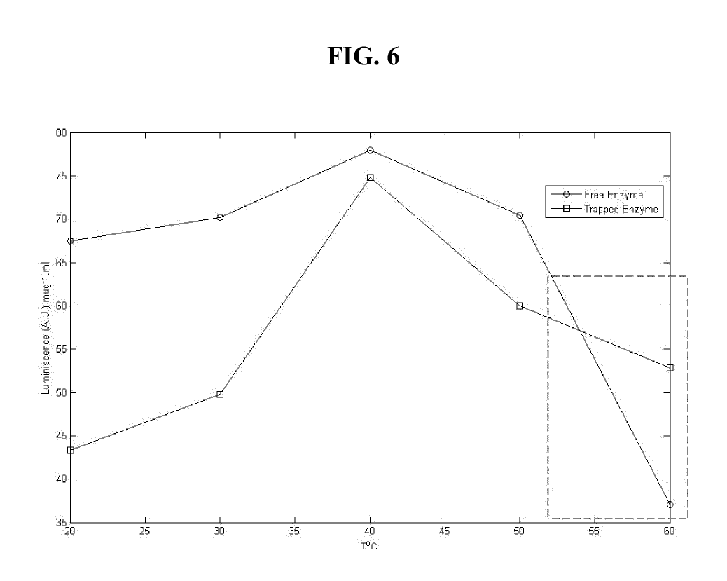

Additionally, the temperature tolerance of nano-cage entrapped luciferase enzyme was tested. At higher temperatures, the luminescence intensity fell for free luciferase enzyme (untrapped luciferase extract) at 40° C. and was absent at 50° C. FIG. 5A-D (left side). However, the entrapment of luciferase enzyme in lipid-functionalized SWCNT reduced the rate of decrease in luciferase enzymatic activity. The luminescence intensity decreased very slowly or essentially remained almost the same up to 40° C. and luminescence still remained at 50° C. FIG. 5A-D (right side). FIG. 6 shows a curve of temperature versus luinescence of trapped versus untrapped luciferase. As can be seen from the figure, trapped luciferase maintains luminescence at higher temperatures (e.g., 55° C.-60° C. or higher) as compared to untrapped luciferase.

The results indicate that the nano-cage allows for more intense luminescence at higher temperatures; without wishing to be bound by theory the results show that the nanocage effectively protects the luciferase enzyme from direct exposure to heat. The results indicate that the luciferase composition of the present technology increases the thermostability of luciferase. In particular, these results show that the luciferase compositions of the present technology are useful in reactions or environments that require luciferase stability at elevated temperatures.

### Example 4

### Method of Using a Luciferase Biosensor

By way of example, but not by limitation, the following is an exemplary method for using a biosensor of the present technology.

In some embodiments, a luciferase biosensor could be used to measure molecular oxygen in a water sample, a gas sample (e.g., from a subjects breath or from the air), dissolve molecular oxygen in soft solids (e.g., tissue) or gels, or atmospheric oxygen (e.g., at high altitude).

In some embodiments, the biosensor is place into the sample (e.g., placed in a stream), or the sample is exposed to the biosensor (e.g., breathing onto the biosensor or dropping sample liquid onto the biosensor).

In some embodiments, the sensor would provide a direct readout of oxygen concentration in an arbitrary unit. In some embodiments, the unit has a conversion chart or calibration table to standardize the oxygen reading. In some embodiments, the conversion charts or calibration table will depend on the sample being tested, e.g., blood versus water or a gas sample versus a person's breath.

### EQUIVALENTS

The present invention is not to be limited in terms of the particular embodiments described in this application, which are intended as single illustrations of individual aspects of the invention. Many modifications and variations of this invention can be made without departing from its spirit and scope, as will be apparent to those skilled in the art. Functionally equivalent methods and apparatuses within the scope of the invention, in addition to those enumerated herein, will be apparent to those skilled in the art from the foregoing descriptions. Such modifications and variations are intended to fall within the scope of the appended claims. The present invention is to be limited only by the terms of the appended claims, along with the full scope of equivalents to which such claims are entitled. It is to be understood that this invention is not limited to particular methods, reagents, compounds compositions or biological systems, which can, of course, vary. It is also to be understood that the terminology used herein is for the purpose of describing particular embodiments only, and is not intended to be limiting.

1. A method of measuring molecular oxygen, the method comprising: contacting a first sample with at least one luciferase composition, wherein the luciferase composition comprises at least one lipid-functionalized graphene or lipid-functionalized fullerene and at least one luciferase, wherein the luciferase is entrapped within the lipid-functionalized graphene or the lipid-functionalized fullerene, wherein the entrapped luciferase is not linked to the lipid-functionalized graphene or the lipid-functionalized fullerene; and detecting luminescence.

2. The method of claim 1, wherein the lipid-functionalized fullerene comprises at least one lipid-functionalized carbon nanotube.

3. The method of claim 2, wherein the lipid-functionalized carbon nanotube comprises one or more of single-wall carbon nanotubes (SWCNT), double-walled carbon nanotubes, or multi-walled carbon nanotubes.

4. The method of claim 1, wherein relative intensity of the luminescence correlates to the amount of molecular oxygen in the sample.

5. The method of claim 1, further comprising comparing the luminescence to at least one control.

6. The method of claim 1, further comprising measuring molecular oxygen, wherein measuring molecular oxygen comprises calibrating the luminescence against an oxygraph reading, using a Clarke electrode, using REDFLASH-dyes, or using optical oxygen sensing based on ruthenium and porphyrin complexes.

7. The method of claim 1, wherein the luciferase is a bacterial luciferase.

8. The method of claim 7, wherein the bacterial luciferase comprises one or more luciferase from Vibrio fischeri, Photobacterium species, Vibrio harveyi, Photobacterium leiognathi, Vibrio logei, Photorhabdus sp., Alteromonas sp., or a combination thereof.

9. The method of claim 1, wherein the luciferase is a non-bacterial luciferase.

10. The method of claim 9, wherein the non-bacterial luciferase comprises one or more luciferase from Renilla luciferase, firefly luciferase, or a combination thereof.

11. The method of claim 1, wherein the luciferase is a recombinant luciferase.

12. The method of claim 1, wherein the luciferase composition further comprises at least one adsorbtion agent, scavenging agent, or a combination thereof.

13. The method of claim 12, wherein the adsorption agent comprises one or more of melanin particles (Cope), semi-conductor particles, or a combination thereof.

14. The method of claim 12, wherein the scavenging agent comprises one or more anti-oxidants, vitamin C, vitamin E, glutathione, or a combination thereof.

15. The method of claim 1, wherein the luciferase composition further comprises an associated protein, wherein the associated protein is entrapped within the lipid-functionalized graphene or the lipid-functionalized fullerene, wherein the entrapped associated protein is not linked to the lipid-functionalized graphene or the lipid-functionalized fullerene.

16. The method of claim 15, wherein the associated protein comprises NADPH dehydrogenase, lumazine proteins, NADPH-FMN oxidoreductase, yellow fluorescent protein, superoxide dismutase, peroxidases, and fatty acid reductase complex.

17. The method of claim 1, further comprising:
separating the luciferase composition from the first sample; and
contacting a second sample with the luciferase composition.

18. The method of claim 17, further comprising separating the luciferase composition from the second sample and contacting the luciferase composition with a third sample.

19. The method claim 1, wherein the contacting occurs at a temperature between about 40° C. to about 60° C.

20.-77. (canceled)

78. A biosensor device for measuring oxygen comprising:
at least two measuring chambers comprising tops, wherein the measuring chamber contains a mixture of compounds, wherein the mixture of compounds comprises at least one lipid-functionalized graphene or lipid-functionalized fullerene and at least one luciferase, wherein the luciferase is entrapped within the lipid-functionalized graphene or the lipid-functionalized fullerene, wherein the entrapped luciferase is not linked to the lipid-functionalized graphene or the lipid-functionalized fullerene;
at least two removable vessels for receiving a sample, wherein the vessels have a removable top and a needle positioned on a bottom, wherein the vessel and the measuring chamber can be connected by inserting the needle into the top of the measuring chamber;
a housing;
a sample holder positioned within the housing, wherein the measuring chamber can be inserted into the sample holder;
a transparent lens positioned on the sample holder;
a photon detector positioned within the housing;
a signal processor, positioned within the housing, wherein the photon detector and signal processor are connected; and
a display unit connected to the signal processor.

79. A hand-held biosensor device for measuring oxygen comprising:
a catheter;
an optic fiber, wherein a first end of the optic fiber is within the catheter;
a semi-permeable membrane chamber, wherein the semi-permeable membrane chamber is attached to first end of the optic fiber;
a mixture of compounds, wherein the mixture of compounds comprises:
at least one lipid-functionalized graphene or lipid-functionalized fullerene and at least one luciferase, wherein the luciferase is entrapped within the lipid-functionalized graphene or the lipid-functionalized fullerene, wherein the entrapped luciferase is not linked to the lipid-functionalized graphene or the lipid-functionalized fullerene and wherein the mixture is disposed within the semi-permeable membrane chamber;
a photon detector; wherein a second end of the optic fiber is connect to the photon detector; and
a processing unit, wherein the processing unit is connected to the photon detector.

80. The device of claim 79, wherein the semi-permeable membrane chamber is removable.

81. The device of claim 79, wherein the semi-permeable membrane chamber comprises an optode.
